(19) United States
(12) Patent Application Publication
(10) Pub. No.: US 2025/0362260 A1
(43) Pub. Date: Nov. 27, 2025

(54) AMMONIA GAS CONCENTRATION MEASUREMENT METHOD, SEMICONDUCTOR AMMONIA GAS CONCENTRATION MEASUREMENT APPARATUS, AND LIVESTOCK BARN ENVIRONMENT MANAGEMENT METHOD

(71) Applicant: Alps Alpine Co., Ltd., Tokyo (JP)

(72) Inventors: Satoshi WAGA, Tokyo (JP); Masahiko ISHIZONE, Tokyo (JP)

(21) Appl. No.: 19/289,852

(22) Filed: Aug. 4, 2025

Related U.S. Application Data
(63) Continuation of application No. PCT/JP2024/003477, filed on Feb. 2, 2024.

(30) Foreign Application Priority Data
Mar. 10, 2023 (JP) 2023-037987

(51) Int. Cl. G01N 27/12; G01N 33/00

(52) U.S. Cl. CPC G01N 27/125; G01N 33/0006

(57) ABSTRACT

An ammonia gas concentration measurement method uses a semiconductor ammonia gas concentration measurement apparatus having a sensor element that detects a change in resistance value occurring by contact of a metal oxide semiconductor with ammonia gas. The method includes a calibration formula creation step of measuring concentrations of ammonia gas in a plurality of known concentrations with the sensor element for each of a plurality of detection targets having different humidity levels and creating a calibration formula showing a relationship between the concentration of ammonia gas and an output value of the sensor element for each of the humidity levels of the detection targets, and a calculation step of calculating the concentration of ammonia gas based on the output value of the sensor element at the time of detection of ammonia gas, the detection humidity, and at least one calibration formula selected from a plurality of the calibration formulae.

### CLAIM OF PRIORITY

This application is a Continuation of International Application No. PCT/JP2024/003477 filed on Feb. 2, 2024, which claims benefit of Japanese Patent Application No. 2023-037987 filed on Mar. 10, 2023. The entire contents of each application noted above are hereby incorporated by reference.

### BACKGROUND OF THE INVENTION

### 1. Field of the Invention

The present invention relates to an ammonia gas concentration measurement method and a semiconductor ammonia gas concentration measurement apparatus. The concentration measurement method and concentration measurement apparatus of the present invention detect, as a gas concentration, a change in resistance value that occurs when ammonia gas in a detection target and a metal oxide semiconductor are brought into contact with each other.

### 2. Description of the Related Art

A semiconductor sensor element includes a gas-sensing part of which the main component is a metal oxide semiconductor, and when gas in a detection target is brought into contact with the gas-sensing part, the metal oxide semiconductor reacts with the gas to cause electron exchange. Since the resistance value of the metal oxide semiconductor varies by this electron exchange, a gas sensor including a semiconductor gas detection element can extract a change in the resistance value of the metal oxide semiconductor as a sensor output to detect the gas in the detection target.

Japanese Unexamined Patent Application Publication No. 2008-241430 (“PTL 1”) describes a semiconductor gas detection element including a gas-sensing part and a catalyst layer coating the gas-sensing part in order to provide a semiconductor gas detection element having gas selectivity over a long period of time. The catalyst layer in the semiconductor gas detection element of PTL 1 contains a metal complex oxide obtained by dissolving a specific metal element in a metal oxide semiconductor including a specific metal oxide.

However, since the semiconductor gas detection element described in PTL 1 calculates the concentration of ammonia gas without considering the influence of humidity, it is difficult to accurately measure the concentration of ammonia gas. The present invention provides an ammonia gas concentration measurement method and a semiconductor ammonia gas concentration measurement apparatus that can accurately measure the concentration of ammonia gas.

### SUMMARY OF THE INVENTION

To solve the above-mentioned disadvantages, one embodiment of the present invention provides an ammonia gas concentration measurement method using a semiconductor ammonia gas concentration measurement apparatus which includes a sensor element that detects, as a concentration of gas, a change in resistance value occurring by contact of a metal oxide semiconductor with ammonia gas in a detection target. The method comprises (a) a calibration formula creation step of measuring concentrations of ammonia gas in a plurality of known concentrations with the sensor element for each of a plurality of detection targets having different humidity levels and creating a calibration formula showing a relationship between the concentration of ammonia gas and an output value of the sensor element for each of the humidity levels of the detection targets, and (b) a calculation step of calculating the concentration of ammonia gas based on the output value of the sensor element at the time of detection of the ammonia gas, the detection humidity, and at least one calibration formula selected from a plurality of the calibration formulae.

In the ammonia gas concentration measurement method, a plurality of calibration formulae at different humidity levels are created, and the concentration of ammonia gas is calculated using a calibration formula corresponding to the detection humidity detected at the time of ammonia gas detection. Consequently, the influence of humidity is reduced, and the concentration of ammonia gas can be accurately detected.

The calculation step may calculate the concentration of ammonia gas based on the calibration formula created at the humidity that is the closest to the detection humidity among the plurality of calibration formulae created at the different humidity levels. The influence of humidity on the sensor element can be reduced by calculating the concentration of ammonia gas using a calibration formula created under a humidity condition that is the closest to the detection humidity at the time of detection of the ammonia gas concentration.

In the calculation step, the concentration of ammonia gas may be calculated based on the output value at the time of detection, the detection humidity, and a first calibration formula and a second calibration formula created at two humidity levels among the plurality of calibration formulae. Even if there is a difference between the humidity at which a calibration formula is created and the detection humidity, the influence of humidity is reduced by using a plurality of calibration formulae, and the concentration of ammonia gas can be accurately determined.

The humidity at which the first calibration formula is created may be one that is the closest to the detection humidity among the plurality of humidity levels at which the calibration formulae are created. The humidity at which the second calibration formula is created may be one that is the second closest to the detection humidity among the plurality of humidity levels at which the calibration formulae are created. The detection humidity may be a value between the humidity at which the first calibration formula is created and the humidity at which the second calibration formula is created.

When the concentration of ammonia gas is calculated based on two calibration formulae, since the influence of humidity can be more effectively reduced by using a first calibration formula and a second calibration formula that satisfy the above relationship with the detection humidity, the accuracy of the measurement of the concentration of ammonia gas is improved.

When the detection humidity is designated as HQ, the humidity at which the first calibration formula is created is designated as H1, and the humidity at which the second calibration formula is created is designated H2, in the calculation step, the output value at the time of detection is OQ, the output value at the concentration CQ of ammonia gas in the first calibration formula is O1, and the output value at the concentration CQ of ammonia gas in the second calibration formula is O2. On this occasion, O1 and O2 are determined such that H1, H2, HQ, O1, O2, and OQ satisfy Formula (1) below, and the concentration CQ of ammonia gas when the output value in the first calibration formula is O1 and the output value in the second calibration formula is O2 is defined as the concentration of ammonia gas.

$$( HQ - H 1 ) : ( HQ - H 2 ) = ( O Q - O 1 ) : ( OQ - O 2 ) \qquad ( 1 )$$

According to the above embodiment, the true concentration CQ of the detected ammonia gas can be calculated based on the detection humidity HQ and the output value OQ at the time of detection.

In accordance with one embodiment of the present invention, the semiconductor ammonia gas concentration measurement apparatus includes a heater that can heat the sensor element, and in a case of measuring a plurality of different concentrations of the ammonia gas at a predetermined heating temperature of the heater, the sensor element to be used may have a characteristic that the output decreases as the concentration of ammonia gas increases under a plurality of conditions in which the humidity levels of the detection targets are different.

The semiconductor ammonia gas concentration measurement apparatus includes a heater that can heat the sensor element, and in a case of measuring 20 ppm, 33 ppm, 66 ppm, and 100 ppm of the ammonia gas by setting the heating temperature of the sensor element to 300° C., the sensor element to be used may have a characteristic that the output decreases as the concentration of ammonia gas increases even when the humidity of the detection target is any of 30%, 57%, and 66%.

In the measurement of the concentration of ammonia gas, calibration can be easily and accurately performed by using a sensor element of which the output decreases as the concentration of ammonia gas increases.

The metal oxide semiconductor may contain a tungsten oxide-based metal oxide. The metal oxide semiconductor may contain tungsten trioxide as a metal oxide. Since the linearity of a calibration formula is improved by using the above metal oxide semiconductor, the accuracy of measurement of the concentration of ammonia gas is improved.

The calibration formula creation step creates the calibration formulae by formulating a relationship between a known concentration C of ammonia gas and the output value of the sensor element at the time of detection of the ammonia gas under constant humidity for at least three humidity levels in a humidity range in which the semiconductor ammonia gas concentration measurement apparatus is used, the calculation step calculates, when the measured detection humidity Hm is a value between humidity Hi and humidity Hj that are included in the humidity levels at which the calibration formulae are created in the calibration formula creation step, a relational formula Rm(C) showing a relationship between the concentration of ammonia gas at the measured detection humidity Hm and the output value of the sensor element according to the change in the resistance value based on the calibration formula Ri(C) at the humidity Hi and the calibration formula Rj(C) at the humidity Hj, and in the relational formula Rm(C), the concentration of ammonia gas that gives the output value at the time of the detection of ammonia gas may be calculated as a concentration of the detected ammonia gas.

The calibration formula Ri(C) and the calibration formula Rj(C) are represented by following Formula (2) and Formula (3), where ai, bi, aj, and bj are constants:

$$R ( Hi ) = Ri ( C ) = ai \times C + bi \qquad ( 2 )$$

$$R ( Hj ) = Rj ( C ) = aj \times C + bj \qquad ( 3 )$$

The relational formula Rm(C) at the detection humidity Hm is represented by following Formula (4):

$$Rm ( C ) = A \times C + B \qquad ( 4 )$$

$$where A = ai + [ ( aj - ai ) / ( Hj - Hi ) ] \times ( Hm - Hi ) , and$$

$$B = bi + [ ( bj - bi ) / ( Hj - Hi ) ] \times ( Hm - Hi ) .$$

The concentration Cm of the ammonia gas at the detection humidity Hm may be determined by following Formula (5):

$$Cm = ( R m ( C ) - B ) / A \qquad ( 5 )$$

The semiconductor ammonia gas concentration measurement apparatus comprises an ammonia gas detection sensor including a sensor element that detects, as a gas concentration, a change in resistance value that occurs when a metal oxide semiconductor material is brought into contact with ammonia gas in a detection target and a heater that heats the metal oxide semiconductor material of the sensor element, and a humidity sensor.

The semiconductor ammonia gas concentration measurement apparatus can use the humidity measured with a humidity sensor at the time of concentration measurement for calculation of the concentration of ammonia gas and therefore can accurately measure the concentration of ammonia gas.

The semiconductor ammonia gas concentration measurement apparatus may further include a memory unit of measuring concentrations of ammonia gas in a plurality of known concentrations with the sensor element for each of a plurality of detection targets having different humidity levels and storing a calibration formula showing a relationship between the concentration of ammonia gas and the output value of the sensor element for each of the humidity levels of the detection targets, and a calculation unit of calculating the concentration of ammonia gas based on the output value of the sensor element at the time of detection of the ammonia gas, the detection humidity, and at least one calibration formula selected from a plurality of the calibration formulae.

The semiconductor ammonia gas concentration measurement apparatus can accurately measure the concentration of ammonia gas by using the humidity measured with a humidity sensor at the time of measurement with an ammonia gas detection sensor in the calculation of the concentration of ammonia gas.

According to the present invention, when the concentration of ammonia gas is calculated based on the output of a sensor element, the concentration of ammonia gas can be accurately measured by using the detection humidity at the time of detection of ammonia gas.

### BRIEF DESCRIPTION OF THE DRAWINGS

FIG. 1 is a block diagram of a semiconductor ammonia gas concentration measurement apparatus;

FIG. 2A is a graph showing a relationship between the concentration of ammonia gas and the output of a sensor element;

FIG. 2B is a graph showing a relationship between the concentration of ammonia gas and the output of a sensor element;

FIG. 2C is a graph showing a relationship between the concentration of ammonia gas and the output of a sensor element;

FIG. 2D is a graph showing a relationship between the concentration of ammonia gas and the output of a sensor element;

FIG. 3A is a graph showing a relationship between the heater temperature and the output of the sensor element of FIG. 2A for each atmosphere temperature at a humidity of 30%;

FIG. 3B is a graph showing a relationship between the heater temperature and the output of the sensor element of FIG. 2A for each atmosphere temperature at a humidity of 60%;

FIG. 3C is a graph showing a relationship between the heater temperature and the output of the sensor element of FIG. 2A for each atmosphere temperature at a humidity of 75%;

FIG. 3D is a graph showing a relationship between the heater temperature and the output of the sensor element of FIG. 2B for each atmosphere temperature at a humidity of 30%;

FIG. 3E is a graph showing a relationship between the heater temperature and the output of the sensor element of FIG. 2B for each atmosphere temperature at a humidity of 60%;

FIG. 3F is a graph showing a relationship between the heater temperature and the output of the sensor element of FIG. 2B for each atmosphere temperature at a humidity of 75%;

FIG. 3G is a graph showing a relationship between the heater temperature and the output of the sensor element of FIG. 2C for each atmosphere temperature at a humidity of 30%;

FIG. 3H is a graph showing a relationship between the heater temperature and the output of the sensor element of FIG. 2C for each atmosphere temperature at a humidity of 60%;

FIG. 3I is a graph showing a relationship between the heater temperature and the output of the sensor element of FIG. 2C for each atmosphere temperature at a humidity of 75%;

FIG. 3J is a graph showing a relationship between the heater temperature and the output of the sensor element of FIG. 2D for each atmosphere temperature at a humidity of 30%;

FIG. 3K is a graph showing a relationship between the heater temperature and the output of the sensor element of FIG. 2D for each atmosphere temperature at a humidity of 60%;

FIG. 3L is a graph showing a relationship between the heater temperature and the output of the sensor element of FIG. 2D for each atmosphere temperature at a humidity of 75%;

FIG. 4 is a flow chart of an ammonia gas concentration measurement method;

FIG. 5 is a graph for explaining a calibration formula creation step and a calculation step;

FIG. 6A is a graph showing a relationship between a calibration curve 1, a calibration curve 2, and the detection humidity and the output value of a sensor element when ammonia gas in a detection target is measured;

FIG. 6B is a graph three-dimensionally showing a relationship between a calibration curve 1, a calibration curve 2, and the detection humidity and the output value of a sensor element when ammonia gas in a detection target is measured;

FIG. 7 is a graph for explaining a calibration formula creation step (calibration) in an ammonia gas concentration measurement method;

FIG. 8A is a graph showing the output of a sensor element in an example of measurement in a poultry house;

FIG. 8B is a graph showing the detection humidity in an example of measurement in a poultry house; and

FIG. 8C is a graph showing the results of calculation of the concentration of ammonia gas based on the output of a sensor element and the detection humidity in an example of measurement in a poultry house.

### DESCRIPTION OF THE PREFERRED EMBODIMENTS

### Semiconductor Ammonia Gas Concentration Measurement Apparatus

Hereinafter, embodiments of the present invention will be described with reference to the accompanying drawings.

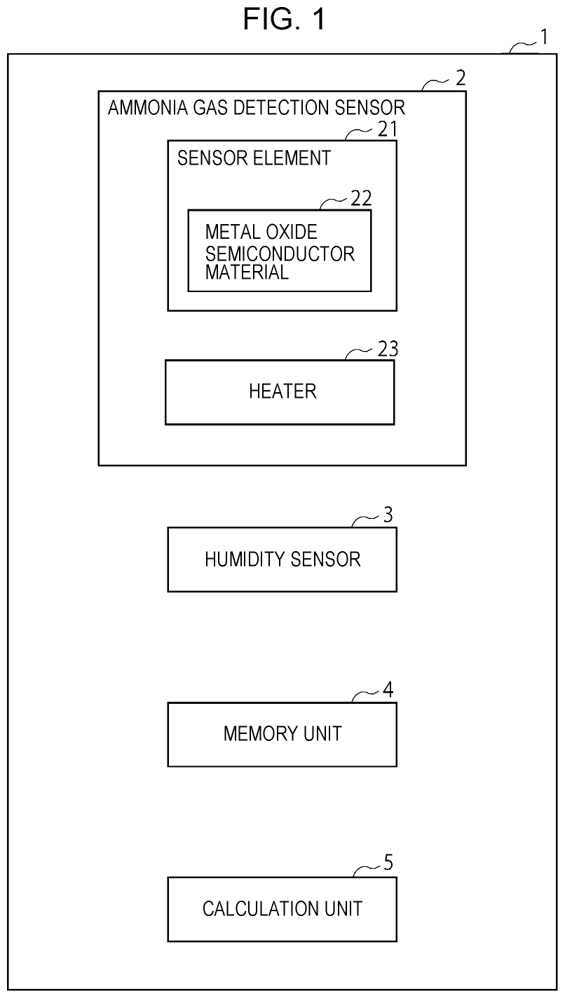

FIG. 1 is a block diagram schematically illustrating a semiconductor ammonia gas concentration measurement apparatus 1 of this embodiment. As is shown in the drawing, the semiconductor ammonia gas concentration measurement apparatus 1 includes an ammonia gas detection sensor 2 and a humidity sensor 3.

The ammonia gas detection sensor 2 includes a sensor element 21 and a heater 23. The sensor element 21 includes a metal oxide semiconductor material 22 and detects, as a gas concentration, a change in resistance value that occurs when the metal oxide semiconductor material 22 is brought into contact with ammonia gas in the detection target.

From the viewpoint of making the sensor element 21 have an output characteristic of high linearity with respect to the concentration of ammonia gas, the metal oxide semiconductor material 22 is preferably a tungsten oxide-based metal oxide and more preferably tungsten trioxide.

The sensor element 21 including the ammonia gas detection sensor 2 is preferably a metal oxide sensor (MEMS-MOx sensor) produced using MEMS (Micro Electro Mechanical Systems) technology of integration by fine processing technology on a substrate such as an electronic circuit.

The MEMS-MOx sensor is produced in a minute region on a thin membrane and therefore has a small heat capacity. Accordingly, a use of the MEMS-MOx sensor as the sensor element 21 has an advantage of decreasing the current value flowing in the heater 23 that heats the sensor element 21. In addition, the MEMS-MOx sensor can be easily integrated with another sensor and a digital signal processing IC into one package and is therefore advantageous in terms of miniaturization of the semiconductor ammonia gas concentration measurement apparatus 1.

The heater 23 is used for heating the metal oxide semiconductor material 22 of the sensor element 21.

The humidity sensor 3 measures the humidity of a detection sample including ammonia gas when ammonia gas that is a detection target of the ammonia gas detection sensor 2 is detected. The humidity sensor 3 is provided based on a finding that the output of a sensor element at the time of detection of ammonia gas varies due to the influence of the humidity of the detection target. The concentration of ammonia gas can be calculated using detection humidity by measuring the detection humidity with the humidity sensor 3 at the time of measurement with the ammonia gas detection sensor 2. Consequently, the influence of the detection humidity on the output of the sensor element 21 is reduced, and the concentration of ammonia gas can be accurately measured.

The semiconductor ammonia gas concentration measurement apparatus 1 of this embodiment includes a memory unit 4 and a calculation unit 5 in addition to the ammonia gas detection sensor 2 and the humidity sensor 3.

The memory unit 4 stores calibration formulae showing a relationship between the concentration of ammonia gas and the output value of the sensor element 21 at a predetermined humidity level, and is a general storage means. The calibration formulae that are stored in the memory unit 4 are created based on the results of measurement of ammonia gas, the concentration of which is known, with the sensor element 21 for each of a plurality of detection targets having different humidity levels under a plurality of conditions in which the concentrations of the ammonia gas are different. The measurement of ammonia gas for creating a calibration formula is performed using the heater 23 of the ammonia gas detection sensor 2 shown in FIG. 1 at a constant temperature.

The calculation unit 5 calculates the concentration of ammonia gas based on the output value of the sensor element 21 at the time of detection of the ammonia gas, the detection humidity, and at least one calibration formula selected from a plurality of the calibration formulae stored in the memory unit 4. The calculation unit 5 is configured as a part of a central processing unit (CPU) or a program.

The creation of the calibration formulae that are stored in the memory unit 4 and the calculation of the concentration of ammonia gas by the calculation unit 5 will be described later as the ammonia gas concentration measurement method.

### Type of Metal Oxide to be Added and Property of Metal Oxide Semiconductor Material

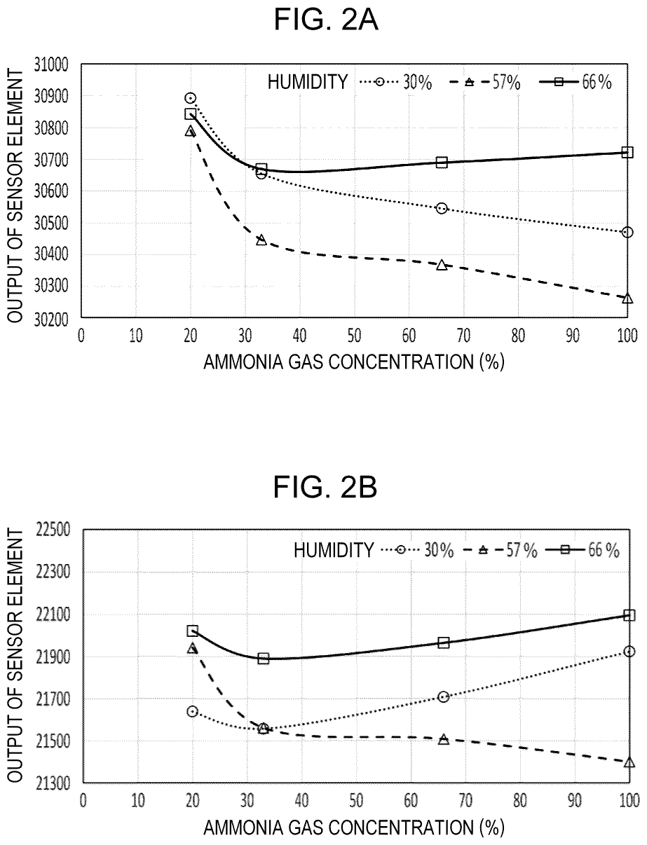

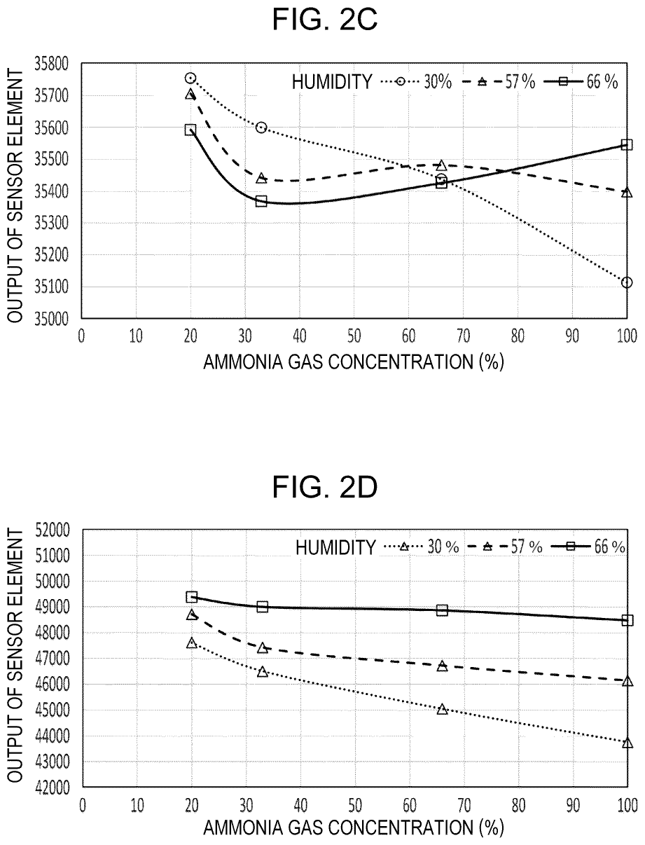

FIGS. 2A to 2D are graphs showing a relationship between the concentration of ammonia gas and the output of the sensor element 21 including a metal oxide semiconductor material 22 containing various metal oxides for each humidity level. The metal oxides that are added to the metal oxide semiconductor material 22 are SnO2 and V2O5 in FIG. 2A, In2O3 in FIG. 2B, SnO2 and Pd in FIG. 2C, and WO3 and OsO4 in FIG. 2D. The preset temperature of the heater 23 when ammonia gas is detected by the sensor element 21 was 400° C. in the measurement example shown in FIG. 2A and was 300° C. in the measurement examples shown in FIG. 2B, FIG. 2C, and FIG. 2D. The graphs shown in FIGS. 2A to 2D are examples of measurement results that are necessary for creating calibration formulae in the calibration formula creation step which will be described later.

As are shown in these drawings, the output of the sensor element 21 varies according to changes in the concentration of ammonia gas and is also highly influenced by the humidity in the measurement target containing the ammonia gas. That is, it was demonstrated that when the concentration of ammonia gas is a detection object, the output of the sensor element 21 varies depending on humidity. Accordingly, if the concentration of ammonia gas is calculated based on the output of the sensor element 21 without considering the influence of humidity, it is difficult to accurately measure the concentration of ammonia gas because of the influence of the detection humidity at the time of detection.

Accordingly, the semiconductor ammonia gas concentration measurement apparatus 1 includes, as is shown in FIG. 1, a humidity sensor 3 in addition to the ammonia gas detection sensor 2. Consequently, when the concentration of ammonia gas is calculated, the detection humidity measured with the humidity sensor 3 can be used in addition to the output of the ammonia gas detection sensor 2. Accordingly, the concentration of ammonia gas can be accurately calculated based on the detection humidity at the time of detection of the ammonia gas.

As are shown in FIGS. 2A to 2D, the output characteristics of the sensor element 21 differ based on the type of the metal oxide that is added to the metal oxide semiconductor material 22.

The sensor element 21 shown in FIG. 2D including a metal oxide semiconductor material 22 containing WO3 and OsO4 has a characteristic that the output decreases as the concentration of ammonia gas increases in a case of measuring a plurality of different concentrations of the ammonia gas at a predetermined heating temperature of the heater 23 under a plurality of conditions of different humidity levels of the detection targets, i.e., humidity levels due to the moisture contained as the atmosphere of the ammonia gas in the detection targets. Specifically, when ammonia gas in concentrations of 20 ppm, 33 ppm, 66 ppm, and 100 ppm is measured at a temperature of the heater 23 of 300° C., in all cases where the humidity levels of the detection target are 30%, 57%, and 66%, the output of the sensor element 21 decreases continuously, as the concentration of ammonia gas increases.

That is, the sensor element 21 including a metal oxide semiconductor material 22 containing WO3 and OsO4 has higher linearity of the output with respect to the concentration of ammonia gas than a sensor element 21 including a metal oxide semiconductor material 22 containing another metal oxide. Accordingly, from the viewpoint of accurately measuring the concentration of ammonia gas, a sensor element 21 including a metal oxide semiconductor material 22 containing WO3 and OsO4 is preferable.

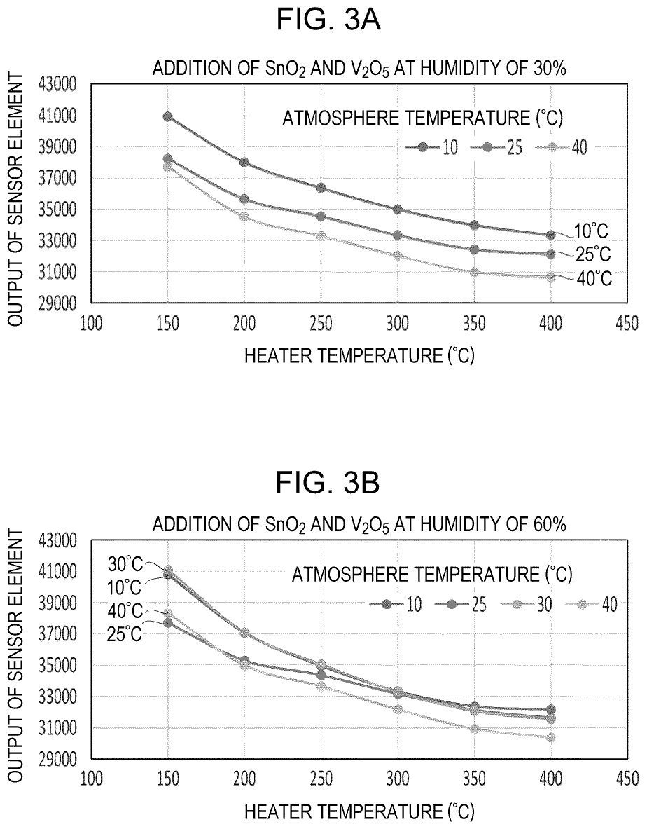

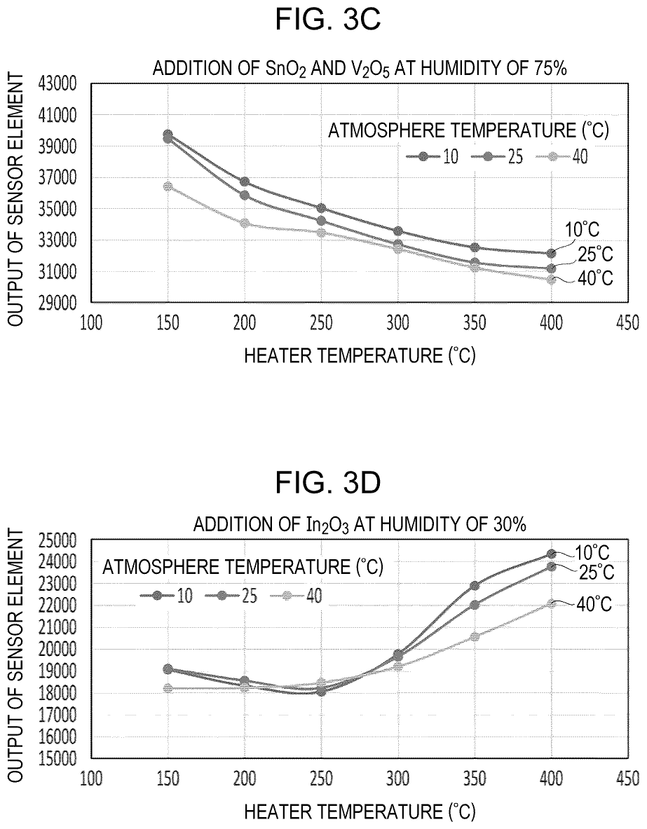

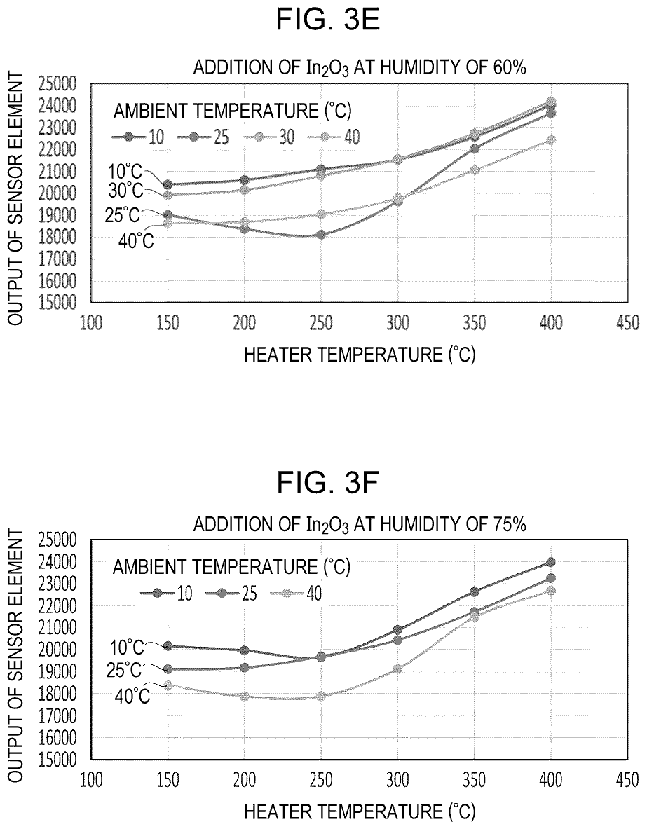

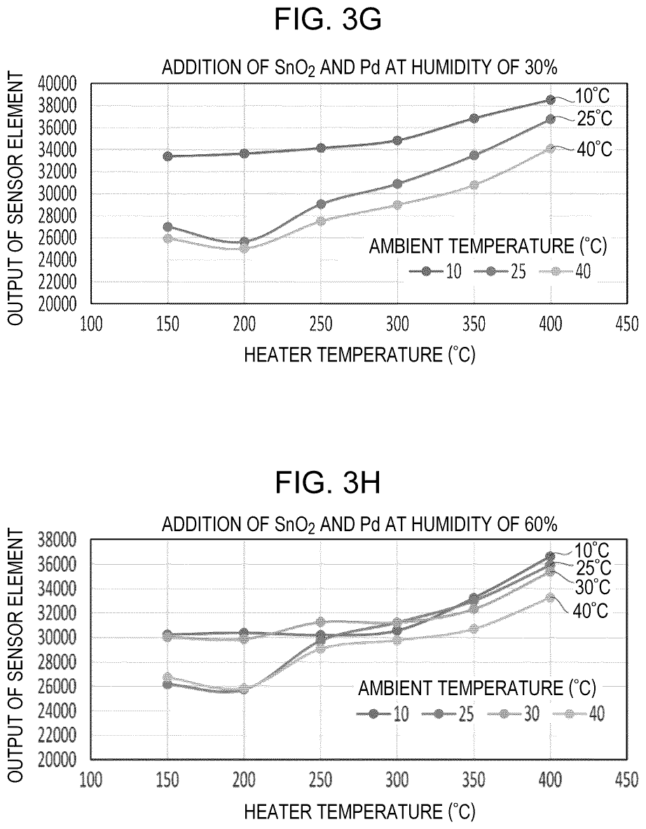

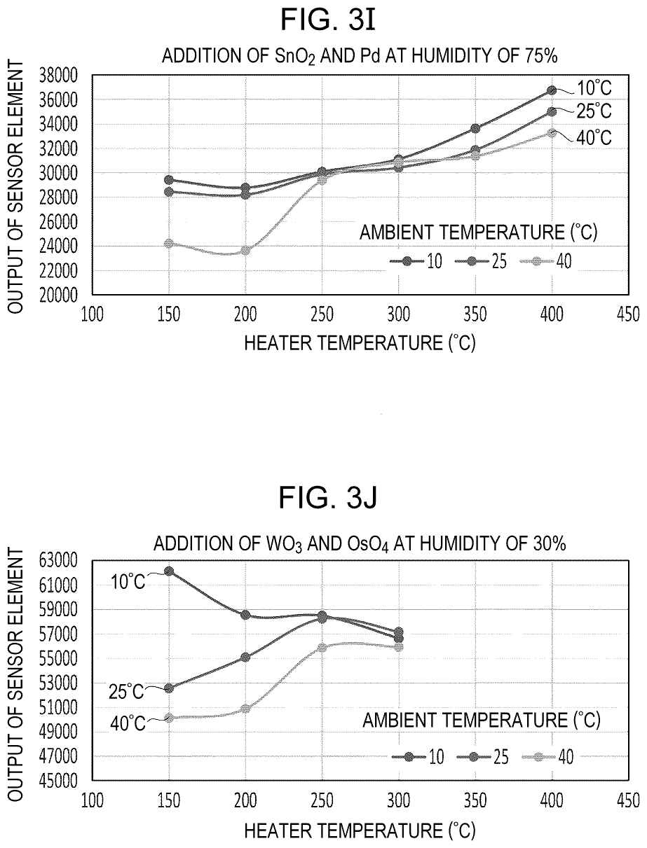

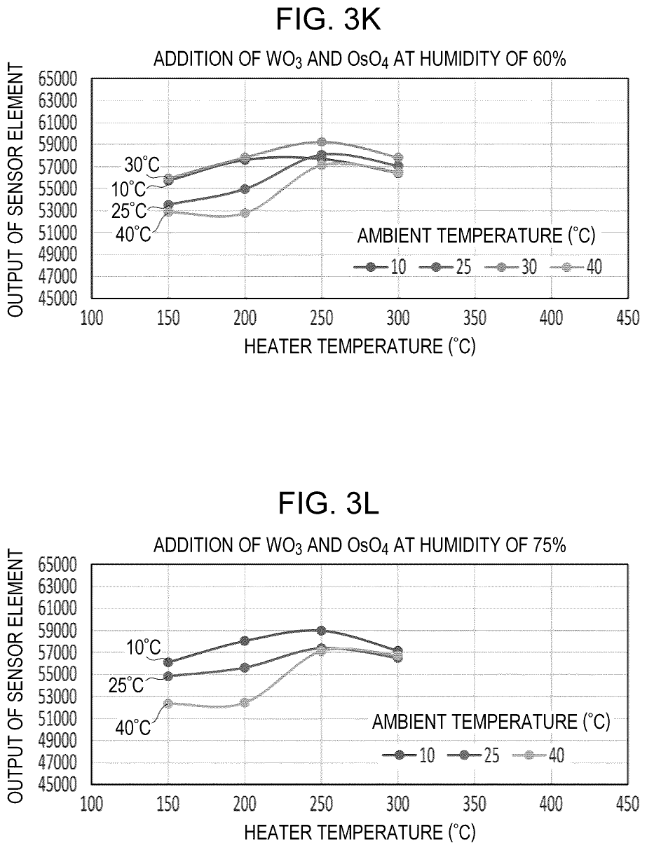

FIGS. 3A to 3L show the results of measurement of the outputs of the sensor elements 21 shown in FIGS. 2A to 2D by changing the temperature of the heater 23 at humidity levels of 30%, 60%, and 75% in a constant-temperature and constant-humidity chamber without ammonia gas. The influences of the humidity and atmosphere temperature on the output of the sensor element 21 were the lowest when the sensor element 21 including a metal oxide semiconductor material 22 containing WO3 and OsO4 was used at a temperature of the heater 23 of 300° C.

As are shown in FIGS. 3J to 3L, the sensor element 21 shown in FIG. 2D obtained an output of about 57000 by setting the temperature of the heater 23 to 300° C., regardless of the humidity and the atmosphere temperature. Accordingly, from the viewpoint of improving the measurement accuracy by suppressing the influences by humidity and atmosphere temperature on the output, it is preferable to use a sensor element 21 including a metal oxide semiconductor material 22 containing WO3 and OsO4 and to set the temperature of the heater 23 at the time of measurement to 250° C. to 350° C., more preferably 280° C. to 320° C.

Examples of the metal oxide that is added to the metal oxide semiconductor material 22 include MnO2, CuO2, Fe2O3, Cr2O3, MoO3, ZnO, Co3O4, TiO3, and NiO in addition to those used in the measurement shown in FIGS. 2A to 2D.

### Ammonia Gas Concentration Measurement Method

An ammonia gas concentration measurement method using the semiconductor ammonia gas concentration measurement apparatus will be described below.

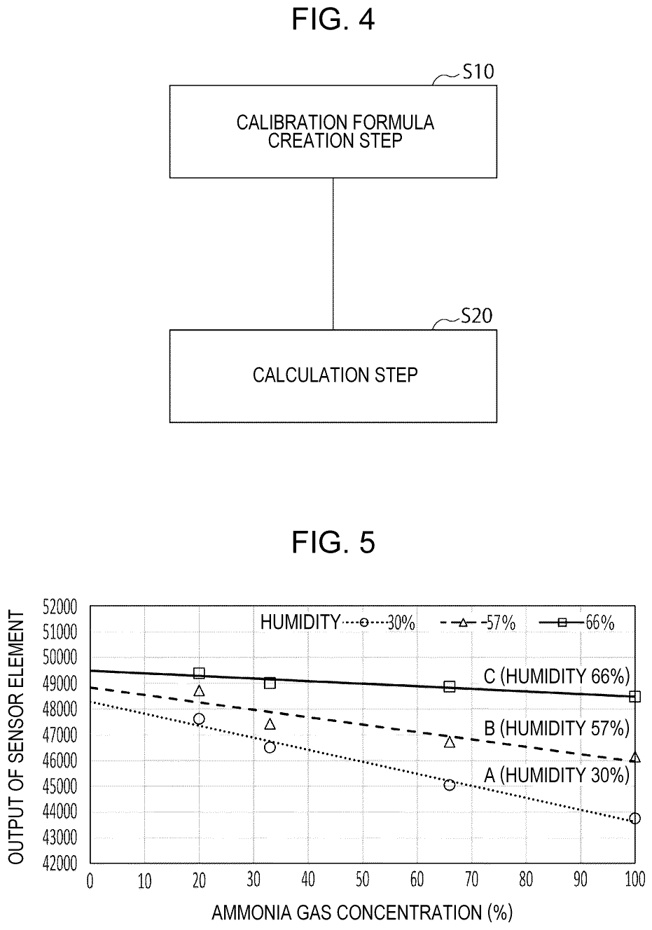

FIG. 4 is a flow chart of the ammonia gas concentration measurement method of the present embodiment. The ammonia gas concentration measurement method includes a calibration formula creation step S10 and a calculation step S20.

In the calibration formula creation step S10, under a plurality of conditions in which the humidity levels of the detection targets are different, various known concentrations of ammonia gas are measured with an ammonia gas detection sensor including a sensor element for each of the humidity levels. Based on the measurement results, a calibration formula showing a relationship between the concentration of ammonia gas and the output of the sensor element is created for each of the humidity levels.

The calibration formula creation step S10 may be performed with only the sensor element (ammonia gas detection sensor) that detects ammonia gas, without performing the step in a state of the semiconductor ammonia gas concentration measurement apparatus. Calibration formulae created by performing the calibration formula creation step S10 with the ammonia gas detection sensor only are stored in the memory unit, and the semiconductor ammonia gas concentration measurement apparatus may be configured using the memory unit in which the calibration formulae are stored, the ammonia gas detection sensor, the humidity sensor, and the calculation unit.

A relationship between the concentration of ammonia gas and the output of the sensor element is obtained by measuring ammonia gas of which the concentration is known with a sensor element. A relationship of the concentration of ammonia gas and the output of the sensor element is obtained by performing this measurement for various known concentrations of ammonia gas is performed under a condition of a constant humidity.

The temperature for creating a calibration formula is the temperature for heating the metal oxide semiconductor material 22 with the heater 23 of the sensor element 21 and is, for example, in a range of about 100° C. to 400° C.

The number of the known different concentrations of ammonia gas to be measured for creating a calibration formula may be 2 or more and is preferably 3 to 5 from the viewpoint of the creation efficiency and accuracy of the calibration curve.

The concentration of ammonia gas to be measured is not particularly limited, but when the outputs of the sensor element are measured at four different concentrations, for example, the concentrations of ammonia gas are 15 to 25 ppm, 28 to 38 ppm, 61 to 71 ppm, and 90 to 100 ppm.

FIG. 5 is a graph for explaining the calibration formula creation step S10 and the calculation step S20 and shows an example of the calibration curve created based on the concentration of ammonia gas and the output of the sensor element of FIG. 2D. In the example shown in the drawing, a relationship between the concentration of ammonia gas and the output of the sensor element is measured at humidity levels of 30%, 57%, and 66%, and a calibration formula is created based on the results of measurement at these humidity levels. A calibration formula expressing a calibration curve can be determined as a regression line by a least-squares method based on the results of measurement at each humidity level.

The calculation step S20 calculates the concentration of ammonia gas based on at least one calibration formula selected from a plurality of calibration formulae, the output value of the sensor element 21 at the time of detection of the ammonia gas, and the detection humidity. A plurality of calibration formulae are those created in the calibration formula creation step S10. The output of the sensor element and the detection humidity that is the humidity detected with the humidity sensor are those obtained by measuring ammonia gas with an unknown concentration.

In the calculation step S20, examples of the method for calculating the concentration of ammonia gas include a method of calculating the concentration of ammonia gas based on a calibration formula created at the humidity that is the closest to the detection humidity among a plurality of calibration formulae created at different humidity levels. The smaller the difference between the humidity condition at the time of creating the calibration formula and the detection humidity, the lower the error of the concentration of ammonia gas from the true concentration of the ammonia gas. Consequently, the influence of humidity on the concentration of ammonia gas is reduced, and the concentration of ammonia gas can be accurately calculated.

In a case of the example shown in FIG. 5, the calibration formula that is used in calculation of the concentration of ammonia gas is the calibration formula at a humidity of 30% when the detection humidity is 38.5% or less, the calibration formula at a humidity of 57% when the detection humidity is greater than 38.5% and 61.5% or less, and the calibration formula at a humidity of 66% when the detection humidity is greater than 61.5%. Thus, the concentration of ammonia gas can be accurately detected based on the influence of humidity by using a calibration formula created at the humidity closest to the detection humidity.

Alternatively, the concentration of ammonia gas may be calculated based on a first calibration formula and a second calibration formula created under different two humidity conditions, not based on one calibration formula. It is possible to more appropriately reflect the influence of the detection humidity on the output value of the sensor element 21 by using a plurality of calibration formulae. Consequently, the accuracy of measurement of the concentration of ammonia gas is improved by calculating the concentration of ammonia gas using two calibration formulae, compared to a case of using one calibration formula.

In the example shown in FIG. 5, for example, calibration formulae at humidity levels of 30% and 57%, calibration formulae at humidity levels of 57% and 66%, or calibration formulae at humidity levels of 66% and 30% may be used in combination.

As is described above, since the smaller the difference between the humidity condition at the time of creating the calibration formula and the detection humidity, the smaller the influence of the humidity, the error of the calculated concentration of ammonia gas from the true concentration of the ammonia gas decreases. In also this case, from the viewpoint of more effectively reducing the influence of humidity, it is desirable to use a calibration formula created at a humidity condition as close as possible to the detection humidity. Based on the above, among various humidity conditions at which calibration formulae have been created, the first calibration formula is preferably the calibration formula at humidity that is the closest to the detection humidity. In addition, among various humidity conditions at which calibration formulae have been created, the second calibration formula is preferably the calibration formula at humidity that is the second closest to the detection humidity. The first calibration formula and the second calibration formula may be selected such that the detection humidity is included between the humidity at which the first calibration formula is created and the humidity at which the second calibration formula is created.

When the measurement results shown in FIG. 5 are used, for example, in a case of a detection humidity of 50%, i.e., humidity between 30% and 57%, the calibration formula at a humidity of 57% that is the closest to the detection humidity of 50% is defined as the first calibration formula, and the calibration formula at a humidity of 30% that is second closest to the detection humidity of 50% is defined as the second calibration formula. When the detection humidity is 48%, a humidity of 48% is the intermediate value between a humidity of 30% and a humidity of 66%. Accordingly, although the detection humidity is between a humidity of 30% and a humidity of 57%, a calibration formula at a humidity of 66% may be used as the second calibration formula.

Since the influence by humidity can be more effectively reduced by using a calibration formula created at humidity that is close to the detection humidity, the accuracy in the measurement of concentration of ammonia gas is improved.

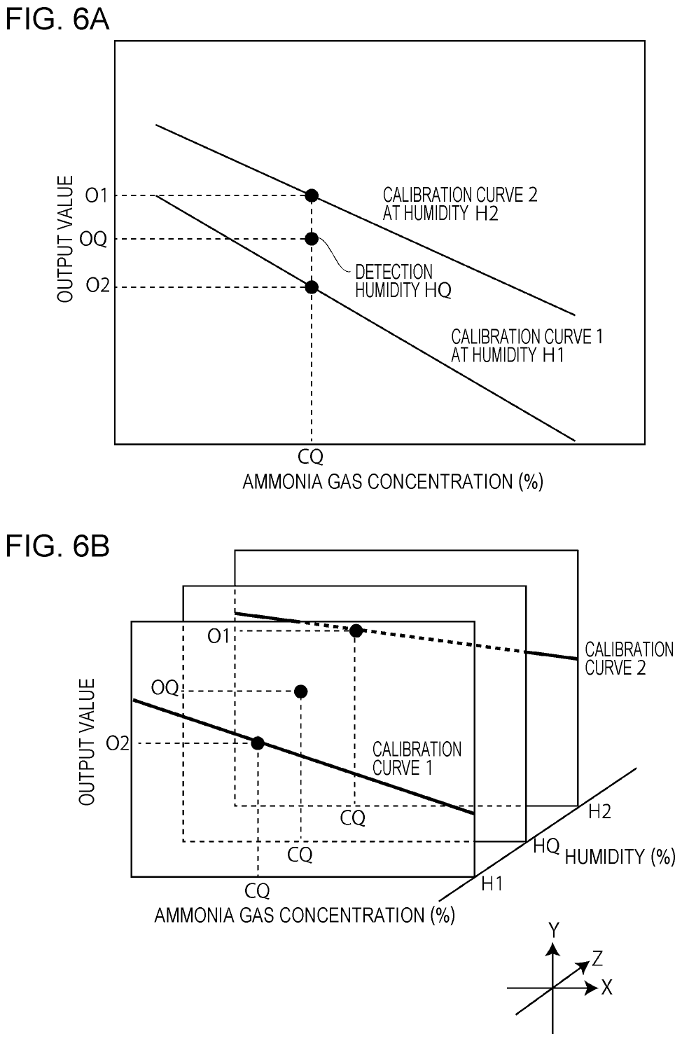

FIG. 6A shows a relationship of a calibration curve 1 determined based on the measurement at humidity H1 and a calibration curve 2 determined based on the measurement at humidity H2 with the detection humidity HQ and the output value OQ of the sensor element when the concentration of ammonia gas in a detection target was measured.

FIG. 6B is a graph showing a relationship among the concentration of ammonia gas, the output value of a sensor element, and the humidity (detection humidity). This graph shows, in orthogonal XYZ coordinates, the concentration of ammonia gas on the X coordinate, the output value of a sensor element on the Y coordinate, and the humidity at the time of calibration curve creation and the humidity at the time of measurement on the Z coordinate.

As is shown in FIG. 6B, it was demonstrated that when the concentration of ammonia gas is constant, the output value of a sensor element varies in a linear form with respect to a change of humidity. The true concentration CQ of the detected ammonia gas can be calculated using this characteristic as follows.

The detection humidity is designated as HQ, the humidity at which the first calibration formula is created is designated as H1, and the humidity at which the second calibration formula is created is designated as H2. In this case, in the calculation step S20, the output value at the time of detection is OQ, the output value at the concentration CQ of ammonia gas in the first calibration formula is O1, and the output value at the concentration CQ of ammonia gas in the second calibration formula is O2. On this occasion, O1 and O2 are determined such that H1, H2, HQ, O1, O2, and OQ satisfy Formula (1) below, and the concentration CQ of ammonia gas when the output value in the first calibration formula is O1 and the output value in the second calibration formula is O2 may be defined as the concentration of ammonia gas:

$$( HQ - H 1 ) : ( HQ - H 2 ) = ( OQ - O 1 ) : ( OQ - O 2 ) . \qquad ( 1 )$$

The true concentration CQ of the detected ammonia gas can be calculated using the above formula (1) based on the detected detection humidity HQ and the output value OQ at the time of detection.

FIGS. 6A and 6B show a case of detection humidity HQ that is between humidity H1 at which a calibration curve 1 is created and humidity H2 at which a calibration curve 2 is created, that is, a case of H1<HQ<H2. However, also in cases of HQ<H1<H2 and H1<H2<HQ, the true concentration CQ of ammonia gas detected using the above relationship can be calculated.

### Modification Example

The calibration formula creation step S10 may create a calibration formula that is a formulated relationship between a known concentration C of ammonia gas and the output value of a sensor element at the time of detection of the ammonia gas under constant humidity at at least three humidity levels in a humidity range in which the semiconductor ammonia gas concentration measurement apparatus is used.

In this case, when the measured detection humidity Hm is a value between the humidity Hi and the humidity Hj that are included in the humidity range in which the calibration formula was created in the calibration formula creation step S10, the calculation step S20 calculates the relational formula Rm(C) showing a relationship between the concentration of ammonia gas and the output value of a sensor element according to the change in the resistance value at the measured detection humidity based on the calibration formula Ri(C) at the humidity Hi and the calibration formula Rj(C) at the humidity Hj. In the relational formula Rm(C), the concentration of ammonia gas corresponding to the output value at the time of detection of ammonia gas is calculated as the concentration of the detected ammonia gas.

For example, in the calibration formula creation step S10, a calibration formula 1, a calibration formula 2, and a calibration formula 3 are created for three humidity levels H1, H2, and H3, respectively, wherein, H1<H2<H3.

In this case, when the detection humidity Hm is H1<Hm<H2, in the calculation step S20, a relational formula Rm(C) showing a relationship between the concentration of ammonia gas at the measured detection humidity Hm and the output value of the sensor element according to the change in the resistance value is calculated based on the calibration formula R1(C) at the humidity H1(Hi) as the calibration formula Ri(C) at the humidity Hi and the calibration formula R2(C) at the humidity H2(Hj) as the calibration formula Rj(C) at the humidity Hj.

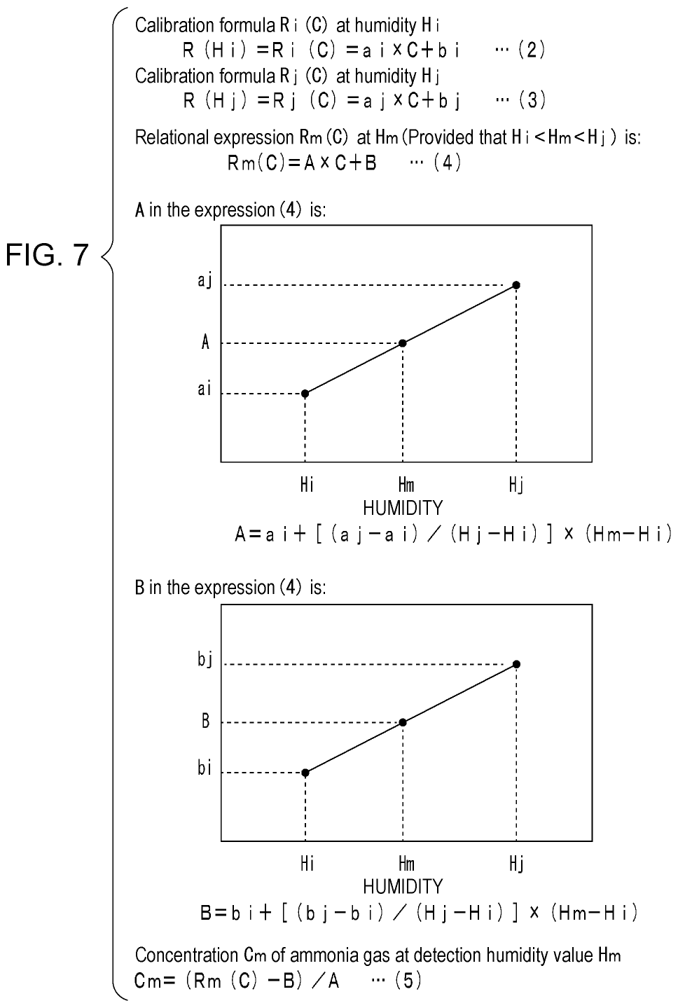

FIG. 7 is an explanation drawing for explaining an ammonia gas concentration measurement method of creating a calibration formula by the method of the modification example and calculating the true concentration of the ammonia gas.

As is shown in the drawing, the calibration formula Ri(C) and the calibration formula Rj(C) represented by Formula (2) and Formula (3) are determined based on the measurement results, where ai, bi, aj, and bj are constants:

$$R ( Hi ) = Ri ( C ) = ai \times C + bi \qquad ( 2 )$$

$$R ( Hj ) = Rj ( C ) = aj \times C + bj \qquad ( 3 )$$

In this case, the relational formula Rm(C) at the detection humidity Hm is represented by following Formula (4) using the constants in the calibration formula Ri(C) and the calibration formula Rj:

$$Rm ( C ) = A \times C + B , \qquad ( 4 )$$

$$where A = ai + [ ( aj - ai ) / ( Hj - Hi ) ] \times ( Hm - Hi ) , and$$

$$B = bi + [ ( bj - bi ) / ( Hj - Hi ) ] \times ( Hm - Hi ) .$$

Accordingly, the concentration Cm of ammonia gas at the detection humidity Hm can be determined by following Formula (5):

$$Cm = ( Rm ( C ) - B ) / A . \qquad ( 5 )$$

### Example of Calibration Curve

Calibration formulae below were obtained using the method of modification example shown in FIG. 7 from the measurement results with a sensor element in which WO3 and OsO4 were added as metal oxides to a metal oxide semiconductor material. The slope was determined by linear approximation, as a result, the values of the intercepts of the humidity levels of 30%, 57%, and 66% were approximately the same, and therefore the intercept of the calibration formula at each humidity level was defined as 57000.

The sensor element that is used in the measurement of the concentration of ammonia gas has the same constitution as the sensor element shown in FIG. 2D and FIG. 5, but they are different individuals. Accordingly, the sensor element used in this measurement example and the sensor element shown in FIG. 2D and FIG. 5 are the same in that they exhibit a characteristic of simply decreasing the output with respect to the concentration of ammonia gas and that the decreasing amount tends to be large in low humidity and small in high humidity, but the intercept values and the slope values of the linear approximations are different.

When the humidity is 30%,

$$R = - 46.611 \times C + 57000 , \qquad ( 6 )$$

when the humidity is 57%,

$$R = - 28.743 \times C + 57000 , \qquad ( 7 )$$

And when the humidity is 66%,

$$R = - 10.022 \times C + 57000 . \qquad ( 8 )$$

The intercepts bi and bj of each calibration formula were defined as 57000, Formula (2) to Formula (5) above0 are represented by following Formulae (2a) to Formula (5a), where ai and aj are constants, and A=ai+[(aj−ai)/(Hj−Hi)]×(Hm−Hi):

$$R ( Hi ) = Ri ( C ) = ai \times C + 57000 \qquad ( 2 a )$$

$$R ( Hj ) = Rj ( C ) = aj \times C + 57000 \qquad ( 3 a )$$

$$Rm ( C ) = A \times C + 57000 \qquad ( 4 a )$$

$$Cm = ( Rm ( C ) - 570 \; 00 ) / A . \qquad ( 5 a )$$

When the detection humidity Hm is between 30% and 57%, that is, in a case of 30%<Hm<57%, Formula (6) above corresponds to Formula (2a), and Formula (7) above corresponds to Formula (3a). When the constants in Formula (6) and Formula (7) are substituted into Formula (4a) and Formula (5a), the results are as follows:

$$Rm ( C ) = A \times C + 57000 \qquad ( 4 b )$$

$$A = - 46.61 + [ ( - 28.743 + 46.6 \; 11 ) / ( 57 - 30 ) ] \times ( Hm - 30 ) , and$$

$$Cm = ( Rm ( C ) - 570 \; 00 ) / A . \qquad ( 5 b )$$

When the detection humidity Hm is between 57% and 66%, that is, in a case of 57%<Hm<66%, Formula (7) above corresponds to Formula (2a), and Formula (8) above corresponds to Formula (3a). When the constants in Formulae (6) and Formula (7) are substituted into Formula (4a) and Formula (5a), the results are as follows:

$$Rm ( C ) = A \times C + 57000 \qquad ( 4 b )$$

$$A = - 28.743 + [ ( - 10.022 + 28.743 ) / ( 66 - 57 ) ] \times ( H m - 57 ) , and$$

$$Cm = ( Rm ( C ) - 570 \; 00 ) / A . \qquad ( 5 b )$$

The ammonia gas concentration measurement method of the present embodiment is based on a finding that the output values of a sensor element obtained as a result of the measurement of the concentration of ammonia gas depends on humidity under a constant atmosphere temperature. In addition, the concentration measurement method corresponds to a “calibration” step of measuring the output of a sensor element with respect to the concentration of ammonia gas in a manufacturing process of a semiconductor ammonia gas concentration measurement apparatus including the sensor element in an environment with controlled temperature and humidity values, determining a correction formula to calculate the concentration of ammonia gas, and storing it in the memory unit of the semiconductor ammonia gas concentration measurement apparatus.

### Measurement Example

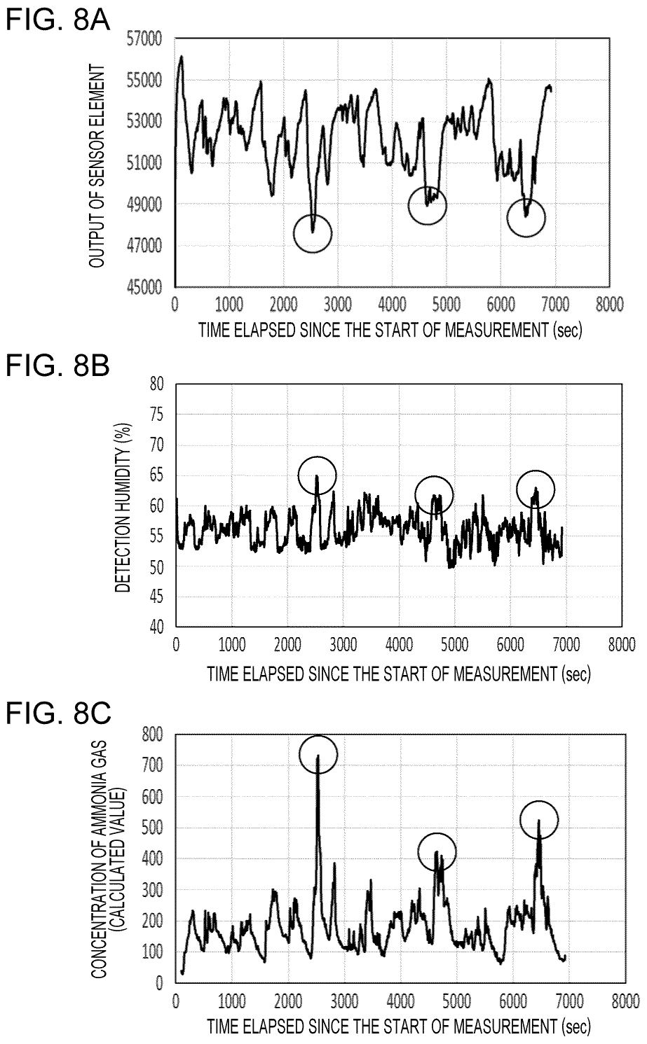

FIG. 8A is a graph showing the output of the sensor element in measurement example in a poultry house of a poultry farm, FIG. 8B is a graph showing the detection humidity in the measurement, and FIG. 8C is a graph showing the concentration of ammonia gas calculated based on the output of the sensor element and the detection humidity in the measurement. These graphs show the results of measurement of the concentration of ammonia gas using a semiconductor ammonia gas concentration measurement apparatus of the present embodiment while walking in the patrol route of six poultry houses over about 7000 seconds. That is, the graphs show the results of the concentration of ammonia gas determined by the ammonia gas concentration measurement method of the present embodiment by acquiring the data of output of the sensor element and the data of detection humidity and performing calculation based on the output of the sensor element and the detection humidity. The concentration of ammonia gas shown in FIG. 8C was calculated using Formula (4b) and Formula (5b).

The calculated value in FIG. 8C used the detection humidity shown in FIG. 8B clearly shows a difference in the concentration of ammonia gas in the poultry house patrol route than the output of the sensor element shown in FIG. 8A. It is demonstrated that there are some points where the concentration of ammonia gas is significantly higher than other measurement points, for example, at the measurement points 2534 seconds, 4725 seconds, and 6454 seconds after the start of measurement shown by circles in the graphs of FIGS. 8A to 8C.

The circled parts of the graphs of FIGS. 8A to 8C are peak values in FIGS. 8B and 8C in contrast with the bottom values in FIG. 8A. This means that the smaller the output value of a sensor element, the higher the concentration of detected ammonia gas and that the higher the humidity, the higher the concentration of ammonia gas.

It is known that the higher the humidity in a poultry house, the higher the concentration of ammonia gas generated from chicken manure, and the results shown in FIGS. 8A to 8C reflect this phenomenon.

Since a means for reducing the concentration of ammonia gas can be taken by specifying a place where the ammonia gas concentration is high in a poultry house, it is possible to promote chicken growth and to improve the working environment in the poultry house.

The accuracy of detection of ammonia gas was improved by detecting also humidity when the concentration of ammonia gas is measured and calculating the concentration of ammonia gas using the detection humidity. Since the concentration of ammonia gas is important for environmental management of a livestock barn such as a poultry house, accurate detection of ammonia gas concentration is useful for maintaining a good livestock barn environment and improving the health of livestock.

The embodiments disclosed in this specification are illustrative in all aspects, and the present invention is not limited to the embodiments. The scope of the present invention is indicated by the claims rather than by the description of the above-mentioned embodiments only and is intended to include all modifications within the meaning and scope equivalent to the claims.

The present invention is useful as an ammonia gas concentration measurement method and a semiconductor ammonia gas concentration measurement apparatus that are used in, for example, management of a livestock barn and so on.

What is claimed is:

1. An ammonia gas concentration measurement method using a semiconductor ammonia gas concentration measurement apparatus including a sensor element that detects, as a concentration of gas, a change in resistance value occurring by contact of a metal oxide semiconductor with ammonia gas in a detection target, wherein the ammonia gas in a detection target is ammonia gas generated from livestock feces in a livestock barn,
the method comprising:
a calibration formula creation step of measuring concentrations of ammonia gas in a plurality of known concentrations with the sensor element for each of a plurality of detection targets having different humidity levels and creating a calibration formula showing a relationship between the concentration of ammonia gas and an output value of the sensor element for each of the humidity levels of the detection targets; and
a calculation step of calculating the concentration of ammonia gas based on the output value of the sensor element at the time of detection of the ammonia gas, the detection humidity, and at least one calibration formula selected from a plurality of the calibration formulae.

2. The ammonia gas concentration measurement method according to claim 1, wherein the calculation step calculates the concentration of ammonia gas based on the calibration formula created at humidity that is the closest to the detection humidity among the plurality of calibration formulae created at the different humidity levels.

3. The ammonia gas concentration measurement method according to claim 1, wherein the calculation step calculates the concentration of ammonia gas based on the output value at the time of detection, the detection humidity, and a first calibration formula and a second calibration formula created at two humidity levels among the plurality of calibration formulae.

4. The ammonia gas concentration measurement method according to claim 3, wherein the humidity at which the first calibration formula is created is the closest to the detection humidity among the plurality of humidity levels at which the calibration formulae are created.

5. The ammonia gas concentration measurement method according to claim 4, wherein the humidity at which the second calibration formula is created is the second closest to the detection humidity among the plurality of humidity levels at which the calibration formulae are created.

6. The ammonia gas concentration measurement method according to claim 3, wherein the detection humidity is a value between the humidity at which the first calibration formula is created and the humidity at which the second calibration formula is created.

7. The ammonia gas concentration measurement method according to claim 3, wherein
when the detection humidity is designated as HQ, the humidity at which the first calibration formula is created is designated as H1, and the humidity at which the second calibration formula is created is designated as H2,
in the calculation step,
the output value at the time of detection is OQ,
the output value at the concentration CQ of ammonia gas in the first calibration formula is O1, and
the output value at the concentration CQ of ammonia gas in the second calibration formula is O2,
O1 and O2 are determined such that H1, H2, HQ, O1, O2, and OQ satisfy a following formula (1), and
the concentration CQ of ammonia gas when the output value in the first calibration formula is O1 and the output value in the second calibration formula is O2 is defined as the concentration of ammonia gas:
$$( HQ - H 1 ) : ( HQ - H 2 ) = ( OQ - O 1 ) : ( OQ - O 2 ) . \qquad ( 1 )$$

8. The ammonia gas concentration measurement method according to claim 1, wherein
the semiconductor ammonia gas concentration measurement apparatus includes a heater that can heat the sensor element, and
in a case of measuring a plurality of different concentrations of the ammonia gas at a predetermined heating temperature of the heater,
under a plurality of conditions in which the humidity levels of the detection targets are different, and
the sensor element to be used has a characteristic that the output decreases as the concentration of ammonia gas increases.

9. The ammonia gas concentration measurement method according to claim 1, wherein
the semiconductor ammonia gas concentration measurement apparatus includes a heater that can heat the sensor element, and
in a case of measuring 20 ppm, 33 ppm, 66 ppm, and 100 ppm of the ammonia gas by setting the heating temperature of the sensor element to 300° C.,
even when the humidity of the detection target is any of 30%, 57%, and 66%, and
the sensor element to be used has a characteristic that the output decreases as the concentration of ammonia gas increases.

10. The ammonia gas concentration measurement method according to claim 1, wherein the metal oxide semiconductor contains a tungsten oxide-based metal oxide.

11. The ammonia gas concentration measurement method according to claim 1, wherein the metal oxide semiconductor contains tungsten trioxide as a metal oxide.

12. The ammonia gas concentration measurement method according to claim 1, wherein
the calibration formula creation step creates the calibration formulae by formulating a relationship between a known concentration C of ammonia gas and the output value of the sensor element at the time of detection of the ammonia gas under constant humidity for at least three humidity levels in a humidity range in which the semiconductor ammonia gas concentration measurement apparatus is used,
the calculation step calculates, when the measured detection humidity Hm is a value between humidity Hi and humidity Hj that are included in the humidity levels at which the calibration formulae are created in the calibration formula creation step, a relational formula Rm(C) showing a relationship between the concentration of ammonia gas at the measured detection humidity Hm and the output value of the sensor element according to the change in the resistance value based on the calibration formula Ri(C) at the humidity Hi and the calibration formula Rj(C) at the humidity Hj, and
in the relational formula Rm(C), the concentration of ammonia gas that gives the output value at the time of the detection of ammonia gas is calculated as a concentration of the detected ammonia gas.

13. The ammonia gas concentration measurement method according to claim 12, wherein
the calibration formula Ri(C) and the calibration formula Rj(C) are represented by following formulae (2) and (3):
$$R ( Hi ) = Ri ( C ) = ai \times C + bi ; \qquad ( 2 )$$
$$R ( Hj ) = Rj ( C ) = aj \times C + bj , \qquad ( 3 )$$
(in the formulae (2) and (3), ai, bi, aj, and bj are constants), the relational formula Rm(C) at the detection humidity Hm is represented by a following formula (4):
$$Rm ( C ) = A \times C + B \qquad ( 4 )$$
$$A = ai + [ ( aj - ai ) / ( Hj - Hi ) ] \times ( Hm - Hi ) , and$$
$$B = bi + [ ( bj - bi ) / ( Hj - Hi ) ] \times ( Hm - Hi ) , and$$
the concentration Cm of ammonia gas at the detection humidity Hm is determined by a following formula (5):
$$Cm = ( Rm ( C ) - B ) / A . \qquad ( 5 )$$

14. A semiconductor ammonia gas concentration measurement apparatus comprising:
an ammonia gas detection sensor including a sensor element that detects, as a gas concentration, a change in resistance value that occurs when a metal oxide semiconductor material is brought into contact with ammonia gas in a detection target and a heater that heats the metal oxide semiconductor material of the sensor element; and
a humidity sensor.

15. The semiconductor ammonia gas concentration measurement apparatus according to claim 14 further comprising:
a memory unit of measuring concentrations of ammonia gas in a plurality of known concentrations with the sensor element for each of a plurality of detection targets having different humidity levels and storing a calibration formula showing a relationship between the concentration of ammonia gas and the output value of the sensor element for each of the humidity levels of the detection targets; and
a calculation unit of calculating the concentration of ammonia gas based on the output value of the sensor element at the time of detection of the ammonia gas, the detection humidity, and at least one calibration formula selected from a plurality of the calibration formulae.

16. A livestock barn environment management method comprising:
acquiring output data of a sensor element and detection humidity data from a humidity sensor using the semiconductor ammonia gas concentration measurement apparatus according to claim 14 while moving in a patrol route set up in a livestock barn;
measuring a concentration of ammonia gas in the livestock barn using the ammonia gas detection sensor, including:
a calibration formula creation step of measuring concentrations of ammonia gas in a plurality of known concentrations with the sensor element for each of a plurality of detection targets having different humidity levels and creating a calibration formula showing a relationship between the concentration of ammonia gas and an output value of the sensor element for each of the humidity levels of the detection targets; and
a calculation step of calculating the concentration of ammonia gas based on the output value of the sensor element at the time of detection of the ammonia gas, the detection humidity, and at least one calibration formula selected from a plurality of the calibration formulae;
specifying a place where the ammonia gas concentration is high in the livestock barn from the measured ammonia gas concentration; and
taking a means for reducing the concentration of ammonia gas in the specified place.
